(19) United States
(12) Patent Application Publication
(10) Pub. No.: US 2019/0009076 A1
(43) Pub. Date: Jan. 10, 2019

(54) PROPHYLACTIC NEURAL STIMULATION

(71) Applicant: BRAINSGATE LTD., Caesarea (IL)

(72) Inventors: Avinoam Dayan, Zichron Yaakov (IL); Yoram Solberg, Hertzeliya (IL); Israel Dvorsky, Kfar Saba (IL)

(21) Appl. No.: 15/641,675

(22) Filed: Jul. 5, 2017

(51) Int. Cl. A61N 1/05; A61N 1/32; A61N 1/36

(52) U.S. Cl. CPC A61N 1/0546; A61N 1/3606; A61N 1/36025; A61N 1/32

(57) ABSTRACT

A method is provided including identifying a subject as being at risk of cognitive decline due to cardiac surgery. The method further includes implanting at least one neural stimulator in a vicinity of a sphenopalatine ganglion (SPG) of the subject, and applying stimulation to the SPG by activating the neural stimulator at least during the cardiac surgery. Other applications are also described.

### FIELD OF THE INVENTION

Some applications of the invention relate generally to medical procedures and implantable devices. More specifically, some applications of the invention relate to the use of electrical devices for implantation in the head.

### BACKGROUND

Despite many advances in modern day cardiac surgery, postoperative cognitive decline among cardiac surgery patients remains a common complication. Neurological impairment following cardiac surgery is a major cause of postoperative morbidity and mortality. Postoperative stroke and cognitive decline (POCD) are believed to be associated with the presence of ischemic lesions originating from an embolism entering the cerebral circulation during cardiac surgery.

U.S. Pat. No. 7,120,489 to Shalev and Gross, which is incorporated herein by reference, describes apparatus for modifying a property of a brain of a patient, including electrodes applied to a sphenopalatine ganglion (SPG) or a neural tract originating in or leading to the SPG. A control unit drives the electrodes to apply a current capable of inducing (a) an increase in permeability of a blood-brain barrier (BBB) of the patient, (b) a change in cerebral blood flow of the patient, and/or (c) an inhibition of parasympathetic activity of the SPG.

U.S. Pat. No. 7,117,033 to Shalev et al., which is incorporated by reference, describes a method for treating a subject, comprising positioning at least one electrode at at least one site of the subject for less than about 3 hours, applying an electrical current to the site of the subject, and configuring the current to increase cerebral blood flow (CBF) of the subject, so as to treat a condition of the subject. The site is selected from the list consisting of: a sphenopalatine ganglion (SPG) of the subject, a greater palatine nerve of the subject, a lesser palatine nerve of the subject, a sphenopalatine nerve of the subject, a communicating branch between a maxillary nerve and an SPG of the subject, an otic ganglion of the subject, an afferent fiber going into the otic ganglion of the subject, an efferent fiber going out of the otic ganglion of the subject, an infraorbital nerve of the subject, a vidian nerve of the subject, a greater superficial petrosal nerve of the subject, and a lesser deep petrosal nerve of the subject.

U.S. Pat. No. 7,561,919 to Shalev et al., which is incorporated by reference, describes apparatus for application to a subject, including an elongated support element having a length of between 1.8 cm and 4 cm, and having proximal and distal ends; and one or more electrodes fixed to the support element in a vicinity of the distal end thereof, and adapted to apply an electrical current to a sphenopalatine ganglion (SPG) of the subject. The apparatus further includes a receiver, fixed to the support element, and electrically coupled to the electrodes; and a wireless transmitter, adapted to be placed in an oral cavity of the subject, and to be wirelessly coupled to the receiver. Other embodiments are also described.

### SUMMARY OF THE INVENTION

In accordance with some applications of the present invention, apparatus and methods are provided for performing prophylactic neural treatment on a subject. Typically, the prophylactic neural treatment comprises applying stimulation to a sphenopalatine ganglion (SPG) of a subject who is at risk of cognitive decline due to cardiac surgery, but typically is not displaying symptoms of cognitive decline. The stimulation is applied in order to inhibit adverse neurological effects of the cardiac surgery and decrease the chance of developing postoperative cognitive decline. In accordance with some applications of the present invention, at least one neural stimulator is implanted in a vicinity of the sphenopalatine ganglion (SPG) and the stimulation is applied to the SPG by activating the neural stimulator at least during the cardiac surgery.

There is therefore provided in accordance with some applications of the present invention, a method including:

identifying a subject as being at risk of cognitive decline due to cardiac surgery;

implanting at least one neural stimulator in a vicinity of a sphenopalatine ganglion (SPG) of the subject; and

applying stimulation to the SPG by activating the neural stimulator at least during the cardiac surgery.

For some applications, identifying the subject, includes identifying the subject as not displaying symptoms of cognitive decline.

For some applications, implanting the at least one neural stimulator includes implanting two neural stimulators, one at each SPG of the subject.

For some applications, applying stimulation to the SPG includes applying the stimulation for at least one day following the cardiac surgery.

For some applications, applying stimulation to the SPG includes applying the stimulation for up to five days following the cardiac surgery.

For some applications, applying stimulation to the SPG includes applying the stimulation only during the cardiac surgery.

For some applications, the method further includes:

monitoring the subject to identify an adverse effect of the stimulation to the SPG; and

discontinuing application of the stimulation to the SPG, if an adverse effect of the stimulation is identified.

For some applications, implanting the at least one neural stimulator includes advancing the at least one neural stimulator through a greater palatine canal of the subject to the SPG of the subject.

There is further provided in accordance with some applications of the present invention, a method including:

identifying a subject as being at risk of cognitive decline due to cardiac surgery;

implanting at least one neural stimulator in a vicinity of a sphenopalatine ganglion (SPG) of the subject;

during the cardiac surgery, using Doppler sonography to detect a presence of an embolism; and

applying stimulation to the SPG by activating the neural stimulator if the embolism is detected, and withholding the application of stimulation to the SPG if an embolism is not detected.

For some applications, identifying the subject, includes identifying the subject as not displaying symptoms of cognitive decline.

For some applications, using Doppler sonography includes using carotid Doppler ultrasound.

For some applications, using Doppler sonography includes using transcranial Doppler sonography.

For some applications, using Doppler sonography includes detecting whether the embolism includes a blood clot, and wherein applying stimulation includes applying stimulation if the blood clot is detected.

For some applications the method further includes, adjusting a parameter of the applying of the stimulation to the SPG in response to detecting the blood clot.

For some applications, using Doppler sonography includes detecting whether the embolism includes an air embolism, and wherein applying stimulation includes applying stimulation if the air embolism is detected.

For some applications the method further includes, adjusting a parameter of the applying of the stimulation to the SPG in response to detecting the air embolism.

For some applications, using Doppler sonography to detect a presence of an embolism includes using Doppler sonography to differentially indicate the presence of a blood clot or an air embolism.

There is further provided in accordance with some applications of the present invention, apparatus including:

a Doppler sonography device configured to detect a presence of an embolism;

an implantable neural stimulator configured for implantation in a vicinity of a sphenopalatine ganglion (SPG) of the subject; and

control circuitry configured to drive the implantable neural stimulator to apply electrical stimulation to the sphenopalatine ganglion (SPG), if the embolism is detected by the Doppler sonography device.

For some applications, the embolism includes a blood clot and wherein the apparatus is configured to detect the presence of the blood clot.

For some applications, the embolism includes an air embolism and wherein the apparatus is configured to detect the presence of the air embolism.

For some applications, the embolism includes an embolism selected from the group consisting of: a blood clot and an air embolism, and wherein the apparatus is configured to differentially indicate the presence of a blood clot or an air embolism.

For some applications, the Doppler sonography device includes a carotid Doppler ultrasound.

For some applications, the Doppler sonography device includes a transcranial Doppler sonography.

For some applications, the control circuitry is configured to withhold the application of stimulation to the SPG if an embolism is not detected.

The present invention will be more fully understood from the following detailed description of applications thereof, taken together with the drawings, in which:

### BRIEF DESCRIPTION OF THE DRAWINGS

FIG. 1 is a schematic illustration of a subject undergoing cardiac surgery, in accordance with some applications of the present invention;

FIGS. 2A and 2B are schematic illustrations of a subject undergoing cardiac surgery, in accordance with respective applications of the present invention; and

FIG. 3 is a flow chart showing steps for prophylactic neural treatment, in accordance with some applications of the present invention.

### DETAILED DESCRIPTION OF EMBODIMENTS

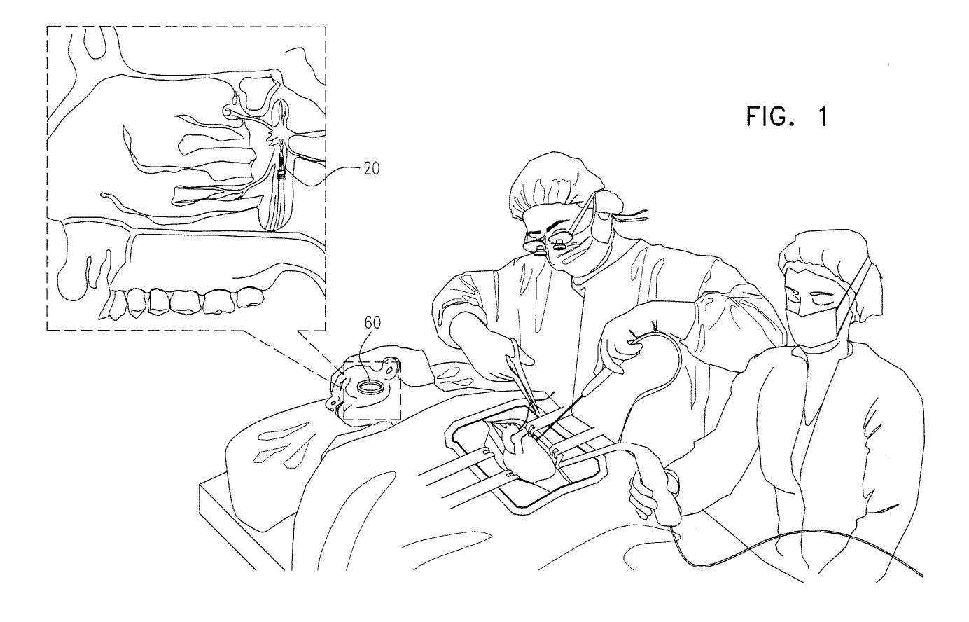

Reference is first made to FIG. 1, which is a schematic illustration of a subject undergoing cardiac surgery, in accordance with some applications of the present invention. Typically, at least one neural stimulator 20 is implanted in a vicinity of a sphenopalatine ganglion (SPG) of the subject who is at risk of cognitive decline due to cardiac surgery but typically is not displaying symptoms of cognitive decline. For some applications, neural stimulator 20 is implanted in a separate, procedure prior to the cardiac surgery. (For example, the neural stimulator may be passed through a greater palatine foramen of a palate of an oral cavity of the subject into a greater palatine canal, until the stimulator is brought into the vicinity of the SPG.). As shown in FIG. 1, a transmitter coil 60, which wirelessly activates implanted stimulator 20, is typically positioned on a face of the subject.

Neural stimulator 20 is activated to apply stimulation to the SPG at least during the cardiac surgery, in order to decrease the chance of the subject developing postoperative cognitive decline. Typically, the neural stimulator comprises at least one electrode which applies an electrical current to the SPG of the subject, causing increased cerebral blood flow (CBF) of the subject. Additionally, or alternatively, implanting the neural simulator in the vicinity of the SPG increases a diameter of a blood vessel, thus decreasing the possibility of obstruction of the blood vessel by a blood clot. As noted hereinabove, neural stimulator 20 is typically prophylactically activated when the subject does not display any symptoms of cognitive decline, in order to inhibit adverse neurological effects of the cardiac surgery.

For some applications, more than one neural stimulator 20, e.g., two neural stimulators are implanted in the subject, such that one neural stimulator is implanted at each SPG of the subject.

For some applications, the stimulation to the SPG is applied only during the cardiac surgery. For other applications, the stimulation to the SPG is applied for at least one day following the cardiac surgery, e.g., the stimulation is applied for up to seven days following the cardiac surgery, but typically for less than five days.

For some applications, following application of the stimulation to the SPG the subject is monitored to identify an adverse effect of the stimulation to the SPG, e.g., a headache, and if such an adverse effect is identified, the stimulation to the SPG is discontinued.

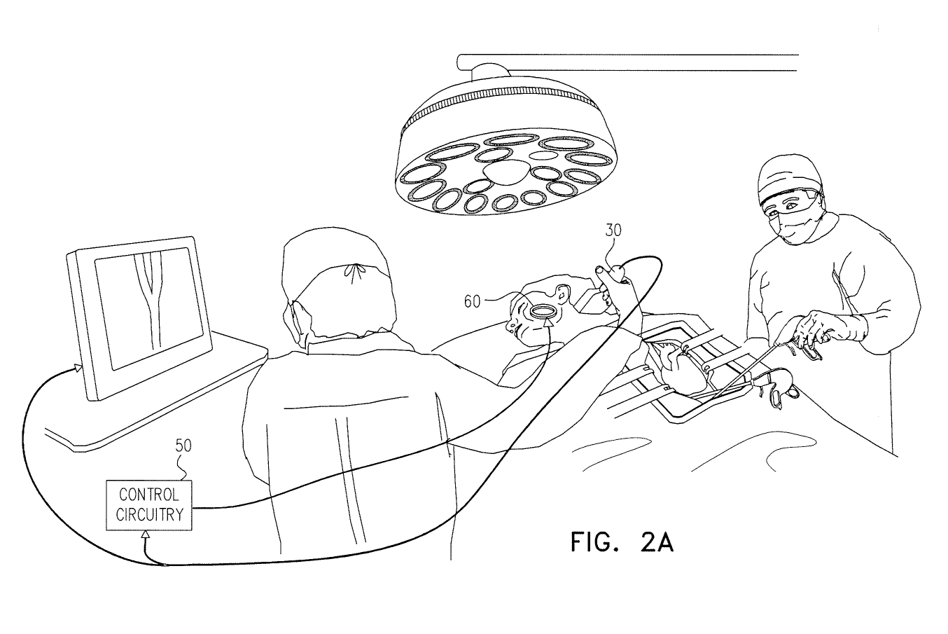

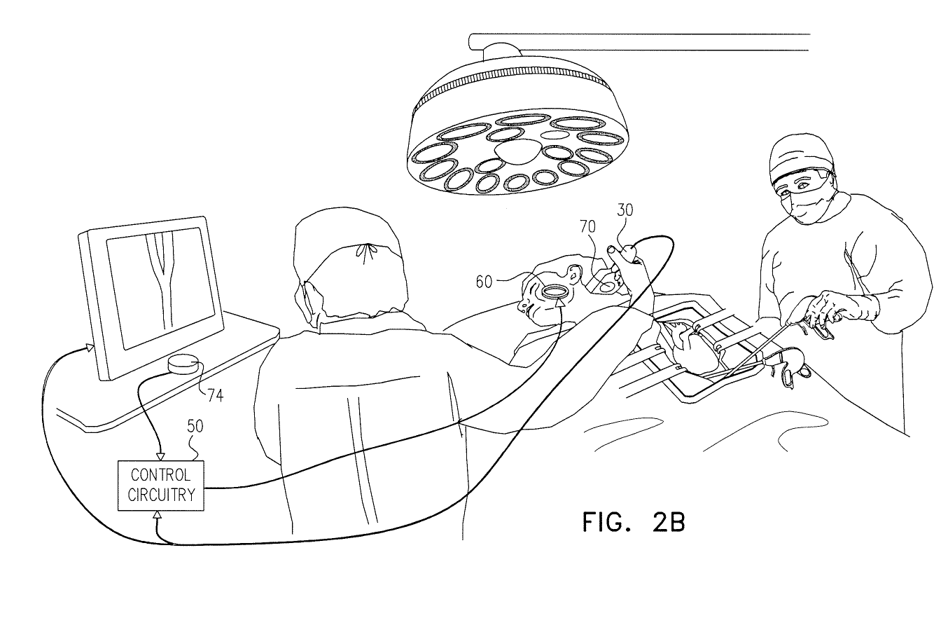

Reference is now made to FIGS. 2A-B, which are schematic illustrations of a subject undergoing cardiac surgery in accordance with some applications of the present invention.

Postoperative cognitive decline is believed to be associated with the presence of ischemic lesions originating from an embolism entering the cerebral circulation during cardiac surgery and causing a stroke. Some applications of the present invention provide using Doppler sonography during the cardiac surgery to detect a presence of an embolism and activating neural stimulator 20 if an embolism is detected. Typically, if an embolism is not detected, activation of the neural stimulator is withheld and no stimulation is applied by the stimulator to the SPG.

As shown in FIGS. 2A-B, a Doppler sonography device 30 is used during the cardiac surgery to assess blood flow through a blood vessel, e.g., a carotid artery, to detect the presence of an embolism that may impair blood flow to and in the brain. It is noted that although FIGS. 2A-B show the surgeon using carotid artery Doppler ultrasound, the scope of the present invention includes using Doppler ultrasound at other locations. For example, transcranial Doppler ultrasound may be used alone or in conjunction with the carotid Doppler ultrasound to measure blood flow through the brain's blood vessels.

Typically, control circuitry 50 drives transmitter coil 60 to activate neural stimulator 20 to apply electrical stimulation to the sphenopalatine ganglion (SPG), if the embolism is detected by the Doppler sonography device. For some applications, such as illustrated in FIG. 2A, control circuitry 50 is configured to automatically drive transmitter coil 60 to activate neural stimulator 20 if an embolism is detected by Doppler sonography device 30. Alternatively, the surgeon manually activates control circuitry 50 to drive transmitter coil 60, e.g., by pressing button 70 or 74 when an embolism is detected by the surgeon (FIG. 2B).

An embolism is typically a blood clot or an air bubble lodged in an artery or a vein causing blockage and inhibiting blood flow in the artery or the vein. In accordance with some applications of the present invention, Doppler sonography device is used to differentially indicate the presence of a blood clot or an air embolism. Typically, neural stimulator 20 is activated in cases of both a blood clot and an air embolism. For example, control circuitry 50 may adjust a parameter of the application of the stimulation to the SPG in response to detecting the embolism. For example, control circuitry 50 may adjust a duration and/or an intensity of the current applied by neural stimulator 20 to the SPG.

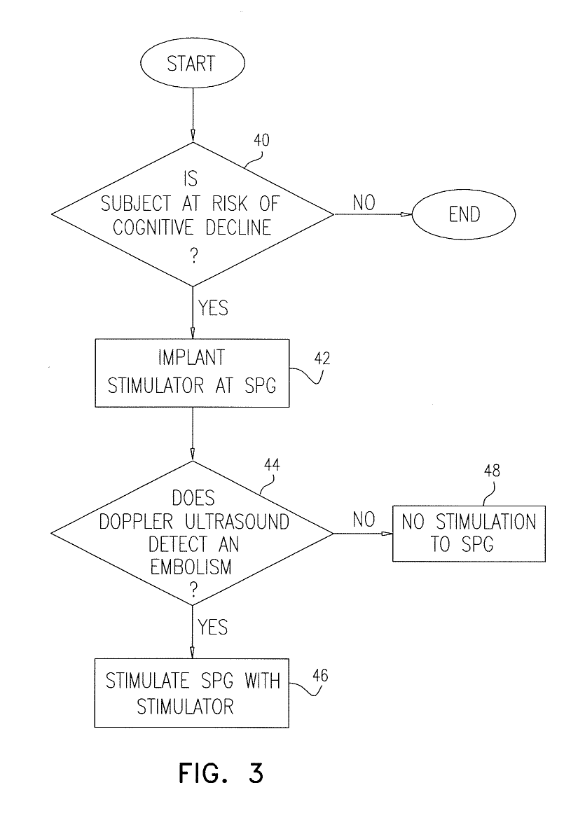

Reference is now made to FIG. 3, which is a block diagram showing steps for prophylactic neural treatment, in accordance with some applications of the present invention. Typically, in step 40, a subject is identified as being at risk of cognitive decline, e.g., a subject undergoing cardiac surgery. A neural stimulator is then implanted at the SPG, as shown in step 42. In step 44, Doppler ultrasound is used to detect an embolism that enters the circulation due to the cardiac surgery, and if an embolism is detected, the neural stimulator is activated to stimulate the SPG (as shown in step 46). If an embolism is not detected, the neural stimulator is not activated and the SPG is not stimulated, as indicated in step 48.

It is noted that in accordance with some applications of the present invention, stimulation to the sphenopalatine ganglion (SPG) is applied to a subject who is at risk of cognitive decline due to any medical condition that increases the risk of a stroke, not limited to cardiac surgery described herein. Accordingly, stimulation is applied to the sphenopalatine ganglion (SPG) of a subject who is at risk of excessive blood clotting which may lead to limited or blocked blood flow. For example, prophylactic stimulation may be applied to the SPG of a subject suffering from diabetes, using apparatus and methods described hereinabove. Diabetes typically increases the risk of plaque buildup in arteries, which can cause blood clots and increase the risk of stroke. Similarly, prophylactic stimulation may be applied to the SPG of a subject suffering from arterial stenosis and/or atrial fibrillation.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method comprising:
identifying a subject as being at risk of cognitive decline due to cardiac surgery;
implanting at least one neural stimulator in a vicinity of a sphenopalatine ganglion (SPG) of the subject; and
applying stimulation to the SPG by activating the neural stimulator at least during the cardiac surgery.

2. The method according to claim 1, wherein identifying the subject, comprises identifying the subject as not displaying symptoms of cognitive decline.

3. The method according to claim 1, wherein implanting the at least one neural stimulator comprises implanting two neural stimulators, one at each SPG of the subject.

4. The method according to claim 1, wherein applying stimulation to the SPG comprises applying the stimulation for at least one day following the cardiac surgery.

5. The method according to claim 4, wherein applying stimulation to the SPG comprises applying the stimulation for up to five days following the cardiac surgery.

6. The method according to claim 1, wherein applying stimulation to the SPG comprises applying the stimulation only during the cardiac surgery.

7. The method according to claim 1, further comprising:
monitoring the subject to identify an adverse effect of the stimulation to the SPG; and
discontinuing application of the stimulation to the SPG, if an adverse effect of the stimulation is identified.

8. The method according to claim 1, wherein implanting the at least one neural stimulator comprises advancing the at least one neural stimulator through a greater palatine canal of the subject to the SPG of the subject.

9. A method comprising:
identifying a subject as being at risk of cognitive decline due to cardiac surgery;
implanting at least one neural stimulator in a vicinity of a sphenopalatine ganglion (SPG) of the subject;
during the cardiac surgery, using Doppler sonography to detect a presence of an embolism; and
applying stimulation to the SPG by activating the neural stimulator if the embolism is detected, and withholding the application of stimulation to the SPG if an embolism is not detected.

10. The method according to claim 9, wherein identifying the subject, comprises identifying the subject as not displaying symptoms of cognitive decline.

11. The method according to claim 9, wherein using Doppler sonography comprises using carotid Doppler ultrasound.

12. The method according to claim 9, wherein using Doppler sonography comprises using transcranial Doppler sonography.

13. The method according to claim 9, wherein using Doppler sonography comprises detecting whether the embolism includes a blood clot, and wherein applying stimulation comprises applying stimulation if the blood clot is detected.

14. The method according to claim 13, further comprising adjusting a parameter of the applying of the stimulation to the SPG in response to detecting the blood clot.

15. The method according to claim 9, wherein using Doppler sonography comprises detecting whether the embolism includes an air embolism, and wherein applying stimulation comprises applying stimulation if the air embolism is detected.

16. The method according to claim 15, further comprising adjusting a parameter of the applying of the stimulation to the SPG in response to detecting the air embolism.

17. The method according to claim 9, wherein using Doppler sonography to detect a presence of an embolism comprises using Doppler sonography to differentially indicate the presence of a blood clot or an air embolism.

18. Apparatus comprising:
a Doppler sonography device configured to detect a presence of an embolism;
an implantable neural stimulator configured for implantation in a vicinity of a sphenopalatine ganglion (SPG) of the subject; and
control circuitry configured to drive the implantable neural stimulator to apply electrical stimulation to the sphenopalatine ganglion (SPG), if the embolism is detected by the Doppler sonography device.

19. The apparatus according to claim 18, wherein the embolism includes a blood clot and wherein the apparatus is configured to detect the presence of the blood clot.

20. The apparatus according to claim 18, wherein the embolism includes an air embolism and wherein the apparatus is configured to detect the presence of the air embolism.

21. The apparatus according to claim 18, wherein the embolism includes an embolism selected from the group consisting of: a blood clot and an air embolism, and wherein the apparatus is configured to differentially indicate the presence of a blood clot or an air embolism.

22. The apparatus according to claim 18, wherein the Doppler sonography device comprises a carotid Doppler ultrasound.

23. The apparatus according to claim 18, wherein the Doppler sonography device comprises a transcranial Doppler sonography.

24. The apparatus according to claim 18, wherein the control circuitry is configured to withhold the application of stimulation to the SPG if an embolism is not detected.
